(19) United States
(12) Patent Application Publication
(10) Pub. No.: US 2018/0235850 A1
(43) Pub. Date: Aug. 23, 2018

(54) COSMETIC PRODUCT AND METHOD FOR PRODUCING THE SAME

(71) Applicant: TOKIWA CORPORATION, Nakatsugawa-shi (JP)

(72) Inventor: Shunsuke Togashi, Kawaguchi-shi (JP)

(73) Assignee: TOKIWA CORPORATION, Nakatsugawa-shi (JP)

(21) Appl. No.: 15/831,399

(22) Filed: Dec. 5, 2017

(30) Foreign Application Priority Data
Feb. 20, 2017 (JP) 2017-029337

(51) Int. Cl. A61K 8/02; A61Q 1/12; A61K 8/34; A61K 8/86; A61K 8/26; A61K 8/73; A61K 8/81; A45D 40/00

(52) U.S. Cl. CPC A61K 8/022; A61Q 1/12; A61K 8/345; A61K 8/86; A61K 2800/48; A61K 8/73; A61K 8/8141; A45D 40/00; A61K 2800/87; A61K 8/26

(57) ABSTRACT

A cosmetic product, and method for producing the same, includes a powder cosmetic layer and a support. The powder cosmetic layer has sufficient adherence to the support, excellent dispensing properties, and a smooth texture during application, and in which generation of color unevenness is sufficiently suppressed. The powder cosmetic layer attached to the support contains a water-soluble component which has a physical property at 25° C. of being is in a liquid state or being a paste, a powder component, and a water-soluble thickener.

### CROSS-REFERENCE TO RELATED APPLICATIONS

This application claims the benefit of priority from Japanese Patent Application No. 2017-029337, filed Feb. 20, 2017, the entire contents of which are incorporated herein by reference.

### FIELD

The present invention relates to a cosmetic product and a method for producing the same, and more specifically to a cosmetic product having a powder cosmetic.

### BACKGROUND

A powder cosmetic such as foundation, eye shadow, blush, and face powder has various forms. In one form, a product is obtained by filling a container with a substance and subsequently compacting and drying the filled substance. The filled substance may contain a dry powder and in some examples, the filled substance may comprise a slurry obtained by kneading the dry powder and a solvent. The dry powder may comprise oil. In other examples, a loose powder-like product is obtained by filling a container with the afore-mentioned substance without compacting the substance.

In order to increase the visual value, such as good appearance, of such a powder cosmetic, in some cases, the shape of a container for cosmetics or the shape of cosmetics themselves are devised. As a contrivance to cosmetics themselves, it is considered that, for example, a powder cosmetic is held on or attached onto a support which is obtained by three-dimensionally molding a base material such as a non-woven fabric. Various methods for holding or attaching a powder cosmetic onto a support have been proposed. One method involves immersing the support in the slurry-like powder cosmetic and drying the powder cosmetic, while another method includes a technique of spraying the powder cosmetic slurry on the support and drying the slurry (for example, Re-Publication WO2012/157643).

### SUMMARY

However, in the powder cosmetic in the related art, the powder cosmetic is easily peeled off from the support, and an adhesion to the support is not sufficient. In a powder cosmetic containing much oil to improve the adhesion to the support, there is a tendency by which appearance or usability is deteriorated due to color unevenness generated on the support, or by which the ease of dispensing or smoothness of texture during application are deteriorated.

An object of the present invention is to provide a cosmetic product having a powder cosmetic which has sufficient adhesion to the support (also referred to herein as adhesion properties) to suitably attach or adhere onto a support, which exhibits a smooth texture, which is easy to dispense (e.g., to pick-up the powder, to lift off the powder from a powder cosmetic layer, or to otherwise transfer the cosmetic product to an applicator such as a brush, to a person's finger, or the like), and which sufficiently inhibits any generation of color unevenness therein, and a method for producing the same.

The present invention for solving the above-described problems provides a cosmetic product including: a support; and a powder cosmetic layer that is held or attached to the support, in which the powder cosmetic layer contains (A) a water-soluble component which is in a liquid state or is a paste at 25° C., (B) powder, and (C) a water-soluble thickener.

The cosmetic product according to the present invention may have a cosmetic layer which is suitably attached to or held by the support and easy to dispense, and exhibits smooth texture during application, and which sufficiently inhibits any generation of color unevenness therein. The powder cosmetic layer exhibits excellent dispensing properties and smooth texture, for example, by using a combination of the components (A) to (C) specified above. In addition, the powder cosmetic layer may be prepared into a slurry form which has a low viscosity and low level of precipitation of powder components when a support is immersed in the powder cosmetic of the powder cosmetic layer which is in a slurry form, and thereby, to uniformly and sufficiently attach the formed powder cosmetic layer to the support.

The present invention also provides a method for producing a cosmetic product, the method including: a step of attaching slurry containing (A) a water-soluble component which is in a liquid state or is a paste at 25° C., (B) powder, (C) the water-soluble thickener, and water, to the support; and a step of forming the powder cosmetic layer containing the water-soluble component, the water-soluble thickener, and the powder by removing a part or the whole of the water from the slurry attached to or held by the support.

According to the method for producing a cosmetic product of the present invention, even if the slurry containing water and the components (A) to (C) specified above has a low viscosity, the powder components nevertheless exhibit a low level of precipitation and the cosmetic product has excellent stability. Therefore, a powder cosmetic layer may be attached to or held by a support which has uniform and sufficient adhesion to the support. Moreover, color unevenness of the formed powder cosmetic layer is sufficiently suppressed, and the powder cosmetic layer is easy to dispense and provides a smooth texture during application.

According to the present invention, a cosmetic product is provided having a powder cosmetic which has sufficient adhesion to the support to attach to a support and which exhibits smooth texture during application, and that is easy to dispense, and sufficiently inhibits any generation of color unevenness therein, and a method for producing the same.

### DESCRIPTION OF THE PREFERRED EMBODIMENTS

Cosmetic products according to the present embodiment include a support and a powder cosmetic layer provided on the support.

In the present invention, the powder cosmetic layer is not limited to a powder cosmetic layer having a clear boundary between a support and the powder cosmetic layer, and includes a powder cosmetic layer which is provided in a state where the support is impregnated with the powder cosmetic layer. In addition, the shape of the powder cosmetic layer may correspond to the shape of the support, but is not particularly limited, and various modifications can be made.

The support is preferably formed of a hydrophilic material. In addition, the support preferably contains an organic compound having a polar group. Specific examples thereof include materials such as cellulose, protein, polyester, polyamide, acryl resin, and polyurethane.

The support can be formed of a film, a woven fabric, a foamed body, a non-woven fabric, and the like which are made of the above-described materials.

From the viewpoint of ease of dispensing of the powder cosmetic, the support may be formed of a film, a woven fabric, or a foamed body. These supports also reduce scuffing of fibers or loss of fibers from the support due to use.

From the viewpoint of adhesion to the support to the powder cosmetic layer, the contact angle of the support to water may be lower than or equal to approximately 90°, or more specifically lower than or equal to approximately 10°. The contact angle to water refers to a value measured by the method described below. In addition, the contact angle may be set to approximately 0° in a case where water permeates the support.

(Method for Measuring Contact Angle) First, water is added in a drop, as a water droplet, to the surface of the support, and an image is obtained by photographing the state with a camera from the sides only. From this image, the contour shape of the droplet is analyzed to calculate the contact angle. The contact angle measured for a droplet is the angle between the surface of the support and a tangent of the contour of the droplet where the droplet meets the surface of the support.

The surface of the support may be subjected to hydrophilic processing such as flame treatment, corona discharge treatment, plasma treatment, silica treatment, flocky processing, and the like.

The shape of the support can be appropriately set according to the use of the cosmetic products. Examples thereof may include three-dimensional shapes such as a hemisphere, a cone, a truncated cone, a pyramid, and a truncated pyramid; shapes of plant parts such as a flower, a flower petal, a fruit, a leaf, and/or any subpart thereof; shapes of other objects such as a sea shell, a jewel, a star shape, and a heart shape; and shapes of parts of a person's face (or other parts of the body), such as the eyelids, the eyes, the lip, and the cheeks, to hold or attach the cosmetic product. These shapes can be prepared, for example, by three-dimensionally molding a sheet-like base material. Examples of the three-dimensional molding method include heat pressing, vacuum molding, and folding, depending on the material of a base material.

The powder cosmetic layer includes (A) a water-soluble component which is formulated to be in a liquid state or is a paste at 25° C. (hereinafter, also referred to as a component (A) in some cases), (B) a powder component (hereinafter, also referred to as a component (B) in some cases), and (C) a water-soluble thickener (hereinafter, also referred to as a component (C) in some cases).

### <Component (A)>

Examples of the water-soluble component which has the consistency of a liquid or a paste at 25° C. include a polyhydric alcohol, a high hydrophilic-lipophilic balance (HLB) surfactant, and a water-soluble oil agent. These components may be used individually or in combination of two or more thereof.

Examples of the polyhydric alcohol include 1,3-butylene glycol, glycerin, propanediol, dipropylene glycol, polyethylene glycol, pentanediol, hexanediol, pentylene glycol, caprylyl glycol, and ethylhexylglycerin.

Examples of the high HLB surfactant include polyglycerin fatty acid ester, fatty acid polyethylene glycol, polyoxyethylene glyceryl ether fatty acid ester, polyoxyethylene hydrogenated castor oil, and polyether modified silicone.

In some examples, the high HLB surfactant is preferably a surfactant having an HLB of approximately 10 to 19 from the viewpoints of suppressing color unevenness of the powder cosmetic layer, and improving makeup durability (e.g., durability of the makeup on a person's skin after being applied) and smoothness of the texture. Examples of such a surfactant include PEG-30 glyceryl isostearate (HLB 15), PEG-10 glyceryl isostearate (HLB 10), polyglyceryl-10 dioleate (HLB 11), PEG-60 hydrogenated castor oil triisostearate (HLB 10), PEG-11 methyl ether dimethicone (HLB 14.5), and PEG-9 dimethicone (HLB 10).

A water-soluble oil agent having a hydroxyl group and/or an ether bond can be used. In a case where the water-soluble oil agent has a hydroxyl group, the hydroxyl group may be included as a glycerin skeleton. In a case where the water-soluble oil agent has an ether bond, the ether bond may be included as a polyethylene glycol skeleton.

Examples of the water-soluble oil agent include polybutylene glycol 3-PEG/PPG-8/5 glycerin, (eicosanedioic acid/tetradecanedioic acid) polyglyceryl-10, polyoxybutylene polyoxyethylene polyoxypropylene methyl glucoside, cyclohexane-1,4-dicarboxylic acid bis(ethoxydiglycol), and methyl gluceth.

The viscosity of the water-soluble component at an approximate room temperature of 25° C. may be in a range of approximately 40 mPa·s to 4,000,000 mPa·s, or approximately 40 mPa·s to 50,000 mPa·s, or approximately 40 mPa·s to 3,000 mPa·s. In some examples, a lower range or value of viscosity may be preferable. Here, the viscosity refers to a value measured with a B-type rotational viscometer.

The amount of the component (A) in relation to the total amount of the powder cosmetic layer may be approximately 1 to 30 mass %, or approximately 1 to 20 mass %. In a case where the amount of the component (A) is greater than or equal to approximately 1 mass %, an adhesion to the support may be increased, color unevenness of the powder cosmetic layer may be reduced (that is to say, the cosmetic powder layer attaches or adheres more effectively and uniformly onto the support), and makeup durability may be increased. In a case where the amount of the component (A) is less than or equal to approximately 30 mass %, the ease of dispensing and the smoothness of the texture of the powder cosmetic layer may be improved

### <Component (B)>

The powder component is not particularly limited as long as it is powder which is usually used in cosmetics. The shape of the powder particles is also not particularly limited. A particle of the powder may have a shape such as a spherical shape, a plate shape, and a needle shape, and may be provided in an amorphous form, a fumed form, a fine particle, a pigment grade particle size, and particle structures such as a porous structure and a non-porous structure. The powder particles may be characterized by any one or multiple ones of the above-mentioned shapes and forms.

Examples of the powder component include brilliant powder such as titanium oxide, ferric ferrocyanide, ultramarine, red oxide, yellow iron oxide, black iron oxide, zinc oxide, aluminum oxide, silica, magnesium oxide, zirconium oxide, magnesium carbonate, calcium carbonate, chromium carbonate, magnesium aluminum hydroxide, mica, synthetic mica, sericite, talc, kaolin, silicon carbide, barium sulfate, silicon nitride, bismuth oxychloride, titanium oxide-coated mica, iron oxide-coated mica, iron oxide-coated mica titanium, organic pigment-coated mica titanium, titanium oxide-coated synthetic mica, titanium oxide-coated glass powder, titanium oxide-iron oxide-coated glass powder, and aluminum powder; organic powder such as nylon powder, polymethyl methacrylate powder, crosslinked polymethyl methacrylate powder, acrylonitrile-methacrylic acid copolymer powder, vinylidene chloride-methacrylic acid copolymer powder, polyethylene powder, polystyrene powder, organopolysiloxane elastomer powder, polymethylsilsesquioxane powder, polyurethane powder, wool powder, silk powder, crystalline cellulose powder, and N-acyl-lysine powder; dye powder such as organic tar pigments and lake pigments of organic dyes; composite powder such as particulate titanium oxide-coated mica titanium, barium sulfate-coated mica titanium, titanium oxide-containing silica, and zinc oxide-containing silica; and metal soap of magnesium stearate, zinc myristate, aluminum stearate, calcium stearate, and the like.

In some example embodiments, the powder cosmetic layer may contain spherical powder which may increase ease of dispensing and improve smoothness of the texture. Examples of spherical powder include silica powder, nylon powder, polyurethane powder, and polymethyl methacrylate powder.

The amount of the spherical powder in relation to the total amount of the powder cosmetic layer may be approximately 1 to 30 mass %, or approximately 1 to 25 mass %. In a case where the amount of the spherical powder is greater than or equal to approximately 1 mass %, the ease of dispensing may be increased and smoothness of the texture of the powder cosmetic layer may be improved. In a case where the amount of the spherical powder is less than or equal to approximately 30 mass %, an adhesion to the support of the powder cosmetic layer may be improved.

The total amount of the component (B) in relation to the total amount of the powder cosmetic layer may be approximately 55 to 98.9 mass %, or approximately 70 to 98.9 mass %.

### <Component (C)>

Examples of the water-soluble thickener include a clay mineral, a polysaccharide, and an acrylic thickener. These may be used individually or in combination of two or more thereof.

Examples of the clay mineral include silicic acid (Al/Mg), bentonite, smectite, montmorillonite, hectorite, silicic acid (Na/Mg), modified bentonite, and synthetic hectorite.

Examples of the polysaccharide include xanthan gum, cellulose gum, locust bean gum, guar gum, carrageenan, gellan gum, starch, dextrin, and alginic acid.

Examples of the acrylic thickener include carbomer, (hydroxyethyl acrylate/acryloyldimethyltaurine Na) copolymer, polyacrylic acid, sodium polyacrylate, crosslinked polyacrylic acid, crosslinked sodium polyacrylate, an acrylic acid-alkyl methacrylate copolymer, and polyacrylamide.

For improving ease of dispensing and smoothness of texture of the powder cosmetic layer, the component (C) may be a clay mineral, an acrylic thickener, or xanthan gum.

The amount of the component (C) in relation to the total amount of the powder cosmetic layer may be approximately 0.1 to 10 mass %, or approximately 0.1 to 5 mass %. In a case where the amount of the component (C) is greater than or equal to approximately 0.1 mass %, the adhesion to the support may be improved, the color unevenness of the powder cosmetic layer may be reduced, and makeup durability may be increased. In a case where the amount of the component (A) is less than or equal to approximately 10 mass %, the ease of dispensing and smoothness of the texture of the powder cosmetic layer may be improved.

Additive components other than the above-described components (A) to (C) may be contained in the powder cosmetic layer.

For example, oil agents, silicones, surfactants, emulsifiers, higher alcohols, natural polymers, synthetic polymers, moisturizers, cool-feeling agents, antiperspirants, ultraviolet absorbers, ultraviolet scattering agents, antioxidants, preservatives, fungicides, pH adjusters, vitamins, blood circulation promoters, whitening agents, skin activators, medicinal ingredients, extracts from animals and plants, dyes, perfumes, and the like may be added to the powder cosmetic layer as the additive components within the scope not impairing the effect of the present invention. However, the present invention is not limited to these examples.

Next, a method for manufacturing a cosmetic product will be described.

The method for manufacturing a cosmetic product includes: a step of preparing slurry which contains a cosmetic base material containing the above-described components (A) to (C), and, as necessary, other additive components, and a dispersion medium containing water; a step of attaching the slurry to a support; and a step of removing a part or the whole of the dispersion medium from the slurry attached to or held by the support.

The slurry may be prepared such that, for example, the component (A) is dissolved in a dispersion medium, and the component (B) is added thereto and is uniformly dispersed therein. Then, the component (C) is gradually added thereto while stirring the mixture. The mixture is further stirred after the addition of the component (C).

The amount of each of the components (A) to (C) in the cosmetic base material may be set in accordance with the above-described ranges of amount in relation to the powder cosmetic layer. In the case where the amount of each of the components (A) to (C) is within the above-described ranges, precipitation of powder components may be reduced, and therefore, the stability of the slurry may be increased.

The formulation ratio of the cosmetic base material containing the components (A) to (C) to the dispersion medium (i.e. cosmetic base material:dispersion medium) may be set to approximately 5:1 by mass ratio, or approximately 2:1 to 1:3 by mass ratio which may increase uniformity of attachment to the support and stability of the slurry.

The amount of water in relation to the total amount of the dispersion medium may be set to approximately 50 to 100 mass %, or approximately 70 to 100 mass % which may increase stability of the slurry.

Aqueous solvents such as ethanol, an isopropyl alcohol, propanol, butanol, and acetone can be used as the dispersion medium other than water. When only water is used, the stability of the slurry may be increased.

An example of a method for mixing the cosmetic base material with the dispersion medium includes a mixing method using a dispersion mixer, a homomixer, a kneader, or the like. In addition, the mixing may be performed while heating the mixture at approximately 15 to 90° C.

As a method for attaching the slurry to the support, one or more of the following methods may be used: a method for immersing the support in the slurry, a method for spraying the slurry on the support, a coating method using a coating tool such as a brush, a writing brush, and a roller, and a method for making the slurry flow from the top of the support so as to make the slurry to be attached to or held by the support.

Removing the dispersion medium from the slurry may be carried out under certain conditions, for example, by exposing the slurry to an ambient temperature within a range of approximately 15 to 100° C. for a time of approximately 10 minutes to 48 hours. The dispersion medium, such as water, may be at least partially evaporated from the slurry. In other examples, a water absorption material or desiccant may be placed in proximity to the slurry for an extended period of time, to cause the slurry to dehydrate. Examples of methods for removing the dispersion medium from the slurry include blow drying, warm air drying, hot air drying, vacuum drying, spin drying, suction drying, and infrared ray-irradiating drying.

Through the above-described processes, a cosmetic product includes a powder cosmetic layer on a support having a predetermined shape, where the powder cosmetic layer has properties to suitably attach to a support and a smooth texture, that is easy to dispense, and in which generation of color unevenness is sufficiently inhibited. In some embodiments, the cosmetic product comprises a plurality of supports and corresponding powder cosmetic layers. For example, the cosmetic product may be shaped like a three-dimensional flower, by shaping each support and powder cosmetic layer in the shape of a flower petal.

The cosmetic product is suitable for blush, face powder, foundation, eye shadow, lipstick, concealer, an eyebrow pencil, temporary hair dye, and the like.

### EXAMPLES

Hereinafter, the present invention will be described in more detail with reference to the following, additional examples.

Prior to the examples, an evaluation method adopted in each example will be described.

### (1) “Ease of Dispensing”, “Makeup Durability”, and “Smoothness of Texture During Application”

20 expert panels for evaluating cosmetic products were allowed to use cosmetic products of examples and comparative examples, and each panel evaluated the cosmetic product in 5 levels regarding “ease of dispensing”, “makeup durability”, and “smoothness of texture” according to the following evaluation criteria. Scores were given for each sample, and an average value of the scores of all of the panels was determined according to the following criteria.

### [Score: Evaluation Criteria]

5 points: very good
4 points: good
3 points: normal
2 points: slightly bad
1 point: bad

### [Criteria (Average Value of Scores)

A: Higher than or equal to 4.5
B: Higher than or equal to 3.5 and lower than 4.5
C: Higher than or equal to 1.5 and lower than 3.5
D: Lower than 1.5

### (2) “Adherence to Support”

A part where the powder cosmetic layer was not provided (a part of a stem of an artificial flower) was tapped 10 times, and a separation condition of the powder cosmetic layer was visually observed and determined according to the following evaluation criteria.

### [Evaluation Criteria]

A: There is almost no separation
B: Slight separation can be seen, but is not noticeable
C: Separation can be seen, and is slightly noticeable
D: Separation can be seen in many portions

### (3) “No Color Unevenness (Color Uniformity)”

The appearance of the powder cosmetic layer was visually observed and determined according to the following evaluation criteria.

### [Evaluation Criteria]

A: No color unevenness is seen
B: Slight unevenness can be seen, but is not noticeable
C: Color unevenness can be seen, and is slightly noticeable
D: Obvious color unevenness can be seen

### (4) “Stability of Slurry”

The stirred slurry was allowed to stand for 30 minutes, and the status was observed and determined according to the following evaluation criteria.

### [Evaluation Criteria]

A: No change
B: Slight supernatant can be seen on the surface
C: Separation can be seen in an upper portion
D: Obvious pigment sedimentation can be seen
(5) “Scuffing of Support and Loss of Fiber from Support”

The status of the support when the cosmetic product was used 50 times with a brush was visually observed and determined according to the following evaluation criteria.

### [Evaluation Criteria]

A: No change
B: Slight scuffing can be seen, but is not noticeable
C: Scuffing is slightly noticeable
D: There is scuffing of the support and loss of fiber from the support

### Examples 1 to 11 and Comparative Examples 1 to 3

A slurry having each composition shown in Tables 1 to 3 was prepared. An artificial flower of each material shown in Tables 1 to 3 was immersed in the slurry and taken out. Thereafter, the artificial flower that was taken out was dried at room temperature for 12 hours or longer to obtain a cosmetic product in which a powder cosmetic layer was formed as blush on the surface of the artificial flower.

The slurry was prepared such that the component (A) or BEHENETH-30 was dissolved in a dispersion medium at 80° C., and the component (B) was added thereto and was uniformly dispersed therein. Then, the component (C) was gradually added thereto while stirring the mixture. The mixture was further stirred after the addition of the component (C).

Artificial flowers formed from polyester fabrics and artificial flowers formed from nylon films were prepared as the supports. The contact angle of the surface of the support to water was measured through the following method.

### <Contact Angle>

Water droplets were added dropwise to the surface of the support, which was photographed using a camera from the sides only. From the acquired image, the contour shapes of the droplets were analyzed to calculate the contact angles.

TABLE 1

| | | | Example | Example | Example | Example | Example | Example |
|---|---|---|---|---|---|---|---|---|
| | | | 1 | 2 | 3 | 4 | 5 | 6 |
| Slurry | Component | 1,3-butylene glycol | 1 | 20 | 30 | 20 | 10 | 10 |
| composition | (A) | Glycerin | — | — | — | — | — | — |
| (parts by mass) | | PEG-30 glyceryl isostearate | — | — | — | — | 2 | — |
| | | (HLB 15) | | | | | | |
| | | PEG-10 glyceryl isostearate | — | — | — | — | — | 2 |
| | | (HLB 10) | | | | | | |
| | | BEHENETH-30(HLB 18) | — | — | — | — | — | — |
| | | (solid) | | | | | | |
| | Component | Silica (spherical powder) | 1 | 25 | 25 | 30 | 10 | 10 |
| | (B) | Mica | 85 | 42 | 32 | 37 | 67.5 | 67 |
| | | Red oxide-coated mica | 5 | 5 | 5 | 5 | 5 | 5 |
| | | Mica titanium | 5 | 5 | 5 | 5 | 5 | 5 |
| | Component | Silicic acid (Al/Mg) | 3 | 3 | 3 | 3 | 0.5 | 1 |
| | (C) | Xanthan gum | — | — | — | — | — | — |
| | | Carbomer | — | — | — | — | — | — |
| | | Cellulose gum | — | — | — | — | — | — |
| | Dispersion | Ion-exchanged water | 100 | 100 | 100 | 100 | 100 | 100 |
| | medium | Isopropyl alcohol | — | — | — | — | — | — |

| | | | | | | | |
|---|---|---|---|---|---|---|---|
| Support | Material | Polyester | Polyester | Polyester | Polyester | Polyester | Polyester |
| | Contact angle (°) | 0 | 0 | 0 | 0 | 0 | 0 |
| Evaluation | Ease of dispensing | A | A | B | A | A | A |
| | Adherence to support | A | A | A | B | B | A |
| | No color unevenness (uniformity of color) | A | A | A | A | B | A |
| | Stability of slurry | A | A | A | A | B | A |
| | Scuffing of support and loss of fiber | A | A | A | A | A | A |
| | Makeup durability | B | A | A | B | B | B |
| | Smoothness of texture during application | B | A | B | A | A | A |

TABLE 2

| | | | Example 7 | Example 8 | Example 9 | Example 10 | Example 11 |
|---|---|---|---|---|---|---|---|
| Slurry | Component | 1,3-butylene glycol | — | 10 | 10 | 10 | 10 |
| composition | (A) | Glycerin | — | 2 | 2 | 2 | — |
| (parts by mass) | | PEG-30 glyceryl isostearate | 5 | — | — | — | — |
| | | (HLB 15) | | | | | |
| | | PEG-10 glyceryl isostearate | — | — | — | — | — |
| | | (HLB 10) | | | | | |
| | | BEHENETH-30(HLB 18) (solid) | — | — | — | — | — |
| | Component | Silica (spherical powder) | 20 | 10 | 10 | 10 | 10 |
| | (B) | Mica | 60 | 58 | 67.9 | 67.5 | 68 |
| | | Red oxide-coated mica | 5 | 5 | 5 | 5 | 5 |
| | | Mica titanium | 5 | 5 | 5 | 5 | 5 |
| | Component | Silicic acid (Al/Mg) | 5 | 10 | — | — | 2 |
| | (C) | Xanthan gum | — | — | 0.1 | — | — |
| | | Carbomer | — | — | — | 0.5 | — |
| | | Cellulose gum | — | — | — | — | — |
| | Dispersion | Ion-exchanged water | 100 | 200 | 100 | 200 | 100 |
| | medium | Isopropyl alcohol | — | — | — | — | — |

| | | | | | | |
|---|---|---|---|---|---|---|
| Support | Material | Polyester | Polyester | Polyester | Polyester | Nylon |
| | Contact angle (°) | 0 | 0 | 0 | 0 | 70 |
| Evaluation | Ease of dispensing | A | B | A | A | A |
| | Adherence to support | A | A | A | A | B |
| | No color unevenness (uniformity of color) | A | A | A | A | B |
| | Stability of slurry | A | A | A | A | A |
| | Scuffing of support and loss of fiber | A | A | A | A | A |
| | Makeup durability | A | A | A | A | A |
| | Smoothness of texture during application | A | B | A | A | A |

TABLE 3

| | | | Comparative | Comparative | Comparative |
|---|---|---|---|---|---|
| | | | Example 1 | Example 2 | Example 3 |
| Slurry | Component | 1,3-butylene | — | 10 | 20 |
| composition | (A) | glycol | | | |
| (parts by | | Glycerin | — | — | — |
| mass) | | PEG-30 glyceryl | — | — | — |
| | | isostearate | | | |
| | | (HLB 15) | | | |
| | | PEG-10 glyceryl | — | — | — |
| | | isostearate | | | |
| | | (HLB 10) | | | |
| | | BEHENETH-30 | 4 | — | — |
| | | (HLB 18) | | | |
| | | (solid) | | | |
| | Component | Silica (spherical | 10 | 10 | 25 |
| | (B) | powder) | | | |
| | | Mica | 74 | 68 | 45 |
| | | Red oxide-coated | 5 | 5 | 5 |
| | | mica | | | |
| | | Mica titanium | 5 | 5 | 5 |
| | Component | Silicic acid | 2 | 2 | — |
| | (C) | (Al/Mg) | | | |
| | | Xanthan gum | — | — | — |
| | | Carbomer | — | — | — |
| | | Cellulose gum | — | 10 | — |
| | Dispersion | Ion-exchanged | 100 | — | 100 |
| | medium | water | | | |
| | | Isopropyl alcohol | — | 100 | — |

| | | | | |
|---|---|---|---|---|
| Support | Material | Polyester | Polyester | Polyester |
| | Contact angle (°) | 0 | 0 | 0 |
| Evaluation | Ease of dispensing | D | A | A |
| | Adherence to support | A | A | D |
| | No color unevenness | A | D | D |
| | (uniformity of color) | | | |
| | Stability of slurry | A | D | D |
| | Scuffing of support and loss | A | A | A |
| | of fiber | | | |
| | Makeup durability | A | A | C |
| | Smoothness of texture during | D | A | A |
| | application | | | |

The details of each component in Tables 1 to 3 are as follows. Glycerin: CONCENTRATED GLYCERIN FOR COSMETICS (manufactured by Kao Corporation, trade name)

PEG-30 glyceryl isostearate: EMALEX GWIS-130 (manufactured by NIHON EMULSION Co., Ltd., trade name)
PEG-10 glyceryl isostearate: EMALEX GWIS-110 (manufactured by NIHON EMULSION Co., Ltd., trade name)
PEG-20 glyceryl triisostearate: EMALEX GWIS-320 (manufactured by NIHON EMULSION Co., Ltd., trade name)
BEHENETH-30: NIKKOL BB-30 (manufactured by Nikko Chemicals Co., Ltd., trade name)
Silica: SUNSIL-130L (manufactured by Sunjin Chemical Co., Ltd., trade name)
Mica: Mica powder Y-2300 (manufactured by YAMAGUCHI MICA CO., LTD., trade name)
Red oxide-coated mica: Colorona Sienna (manufactured by Merck KGaA, trade name)
Mica titanium: Timiron Starluster MP-115 (manufactured by Merck KGaA, trade name)
Silicic acid (Al/Mg): VEEGUM K (manufactured by R.T VANDERBILT CO., INC Ltd., trade name)
Xanthan gum: ECHO GUM (manufactured by DSP GOKYO FOOD & CHEMICAL Co., Ltd., trade name)
Carbomer: AQUPEC HV-505E (manufactured by SUMITOMO SEIKA CHEMICALS CO., LTD., trade name)
Cellulose gum: CMC DAICEL (1160) (manufactured by DAICEL FINECHEM LTD., trade name)

As shown in Tables 1 to 3, the cosmetic products obtained in Examples 1 to 11 were evaluated as “B” or “A” in all categories of “Ease of dispensing”, “Adherence to support”, “No color unevenness (uniformity of color)”, “Stability of slurry”, “Scuffing of support and loss of fiber”, “Makeup durability”, and “Smoothness of texture during application”.

### Example 12: Face Powder

| | (formulation |
|---|---|
| (Component) | ratio (mass %)) |
| 1. Polybutylene glycol 3-PEG/PPG-8/5 glycerin | 5.00 |
| 2. 1,3-butylene glycol | 5.00 |
| 3. Sodium dehydroacetate | 0.30 |
| 4. Simethicone | 0.05 |
| 5. Perfume | 0.15 |
| 6. Mica titanium | 10.00 |
| 7. Synthetic phlogopite | 57.50 |
| 8. Polymethyl methacrylate (spherical powder) | 20.00 |
| 9. Bentonite | 2.00 |
| Dispersion medium: Ion-exchanged water | 100 parts by mass |

with respect to 100 parts by mass in total of the above-described
components 1 to 9

The details of each of the above-described components are as follows.

Component 1: WILBRIDE S-753 (manufactured by NOF CORPORATION, trade name)
Component 4: KS-66 (manufactured by Shin-Etsu Chemical Co., Ltd., trade name)
Component 6: TIMICA SPARKLE (manufactured by BASF Japan Co., Ltd., trade name)
Component 7: PDM-10S (manufactured by TOPY INDUSTRIES LIMITED, trade name)
Component 8: SUNPMMA-S (manufactured by Sunjin Chemical Co., Ltd., trade name)
Component 9: KUNIPIA G (manufactured by KUNIMINE INDUSTRIES CO., LTD., trade name)

### <Production Method>

Components 1 to 3 were dissolved in a dispersion medium, components 4 to 8 were added thereto, and the mixture was uniformly dispersed. Thereafter, a component 9 was gradually added thereto while stirring the mixture. The mixture was further stirred to prepare a slurry. Next, an artificial flower (contact angle of 0°) made of paper was immersed in the slurry and taken out. Thereafter, the artificial flower that was taken out was dried at room temperature for 12 hours to obtain a cosmetic product.

### <Evaluation>

The obtained cosmetic product of face powder was evaluated in the same manner as described above. As a result, it was confirmed that the cosmetic product was evaluated as “A” in all categories of Ease of dispensing, Adherence to support, No color unevenness, Stability of slurry, Scuffing of support and loss of fiber, and Makeup durability.

### Example 13: Foundation

| | (formulation |
|---|---|
| (Component) | ratio (mass %)) |
| 1. (Eicosanedioic acid/tetradecanedioic acid) | 5.00 |
| polyglyceryl-10 | |
| 2. Dipropylene glycol | 5.00 |
| 3. PEG-11 methyl ether dimethicone | 2.00 |
| 4. Sodium dehydroacetate | 0.30 |
| 5. Simethicone | 0.05 |
| 6. Titanium oxide | 4.50 |
| 7. Yellow iron oxide | 0.80 |
| 8. Red oxide | 0.20 |
| 9. Black iron oxide | 0.10 |
| 10. Talc | 20.00 |
| 11. Synthetic phlogopite | 41.55 |
| 12. Nylon powder (spherical powder) | 20.00 |
| 13. (Hydroxyethyl acrylate/acryloyldimethyltaurine | 0.44, 0.03, |
| Na) copolymer, sorbitan isostearate, and | and 0.03 |
| polysorbate 60 | |
| Dispersion medium: Ion-exchanged water | 80 parts by mass |

with respect to 100 parts by mass in total of the above-described
components 1 to 13

| | |
|---|---|
| Ethanol | 20 parts by mass |

with respect to 100 parts by mass in total of the above-described
components 1 to 13

The details of each of the above-described components are as follows.

Component 1: Neosolue-Aqua (manufactured by Nippon Fine Chemical, trade name)
Component 3: KF-6011 (manufactured by Shin-Etsu Chemical Co., Ltd., trade name)
Component 5: KS-66 (manufactured by Shin-Etsu Chemical Co., Ltd., trade name)
Component 6: TIPAQUE PFC407 (manufactured by ISHIHARA SANGYO KAISHA, LTD., trade name)
Component 7: TAROX synthetic iron oxide LL-100P (manufactured by Titan Kogyo, Ltd., trade name)
Component 8: TAROX synthetic iron oxide R-516P (manufactured by Titan Kogyo, Ltd., trade name)
Component 9: TAROX synthetic iron oxide BL-100P (manufactured by Titan Kogyo, Ltd., trade name)
Component 10: Talc JA-13R (manufactured by Asada Milling Co., Ltd., trade name)
Component 11: PDM-5L (manufactured by TOPY INDUSTRIES LIMITED, trade name)
Component 12: SP-500 (manufactured by TORAY INDUSTRIES. INC., trade name)
Component 13: SEPINOV EMT 10 (manufactured by SEIWA KASEI Co., Ltd., trade name)

### <Production Method>

Components 1 to 4 were dissolved in a dispersion medium, components 5 to 12 were added thereto, and the mixture was uniformly dispersed. Thereafter, a component 13 was gradually added thereto while stirring the mixture. The mixture was further stirred to prepare a slurry. Next, a sponge (contact angle of 0°) made of polyurethane was immersed in the slurry and taken out. Thereafter, the artificial flower that was taken out was dried at room temperature for 12 hours to obtain a cosmetic product.

### <Evaluation>

The obtained cosmetic product of foundation was evaluated in the same manner as described above. As a result, it was confirmed that the cosmetic product was evaluated as “A” in all categories of Ease of dispensing, Adherence to support, No color unevenness, Stability of slurry, Scuffing of support and loss of fiber, and Makeup durability.

### Example 14: Eye Shadow

| | (formulation |
|---|---|
| | ratio |
| (Component) | (mass %)) |
| 1. Propanediol | 10.00 |
| 2. Polyglyceryl-10 dioleate (HLB11) | 2.00 |
| 3. PEG-40 hydrogenated castor oil | 2.00 |
| 4. Sodium dehydroacetate | 0.30 |
| 5. Simethicone | 0.05 |
| 6. Red No. 226 | 0.03 |
| 7. Ultramarine | 0.30 |
| 8. Red interference mica titanium | 2.00 |
| 9. Blue interference titanium oxide-coated glass powder | 5.00 |
| 10. Talc | 20.00 |
| 11. Synthetic phlogopite | 46.32 |
| 12. Methyl methacrylate crosspolymer (spherical powder) | 10.00 |
| 13. Silicic acid (Al/Mg) | 2.00 |
| Dispersion medium: Ton-exchanged water | 100 parts by |

mass with respect to 100 parts by mass in total of the above-described
components 1 to 13

| | |
|---|---|
| Ethanol | 20 parts by |

mass with respect to 100 parts by mass in total of the above-described
components 1 to 13

The details of each of the above-described components are as follows.

Component 2: SALACOS PG-218 (manufactured by Nisshin OilliO Group, Ltd., trade name)
Component 3: EMALEX HC-40 (manufactured by NIHON EMULSION Co., Ltd., trade name)
Component 5: KS-66 (manufactured by Shin-Etsu Chemical Co., Ltd., trade name)
Component 6: D&C RED No. 30 (manufactured by Kishi Kasei Co., LTD., trade name)
Component 7: Cosmetic ultramarine blue CB-80 (manufactured by DAIICHI KASEI CO., LTD., trade name)
Component 8: FLAMENCO SPARKLE RED (manufactured by BASF Japan Co., Ltd., trade name)
Component 9: Micro-glass Metashine MT1080RB (manufactured by Nippon Sheet Glass Co. Ltd., trade name)
Component 10: Talc JA-13R (manufactured by Asada Milling Co., Ltd., trade name)
Component 11: PDM-5L (manufactured by TOPY INDUSTRIES LIMITED, trade name)
Component 12: Techpolymer MBX-8C (manufactured by Toray Industries Co., Ltd., trade name)
Component 13: VEEGUM K (manufactured by R.T VANDERBILT CO., INC Ltd., trade name)

### <Production Method>

Components 1 to 4 were dissolved in a dispersion medium, components 5 to 12 were added thereto, and the mixture was uniformly dispersed. Thereafter, a component 13 was gradually added thereto while stirring the mixture. The mixture was further stirred to prepare a slurry. Next, an artificial flower (contact angle of 4°) made of glass was immersed in the slurry and taken out. Thereafter, the artificial flower that was taken out was dried at room temperature for 12 hours to obtain a cosmetic product.

### <Evaluation>

The obtained cosmetic product of eye shadow was evaluated in the same manner as described above. As a result, it was confirmed that the cosmetic product was evaluated as “A” in all categories of Ease of dispensing, Adherence to support, No color unevenness, Stability of slurry, Scuffing of support and loss of fiber, and Makeup durability.

It is to be understood that not all aspects, advantages and features described herein may necessarily be achieved by, or included in, any one particular example embodiment. Indeed, having described and illustrated various examples herein, it should be apparent that other examples may be modified in arrangement and detail. We claim all modifications and variations coming within the spirit and scope of the subject matter claimed herein.

What is claimed is:

1. A cosmetic product comprising:
a support; and
a powder cosmetic layer attached to the support,
wherein the powder cosmetic layer contains:
a water-soluble component having a physical property at 25° C. of being in a liquid state or being a paste,
a powder component, and
a water-soluble thickener.

2. The cosmetic product according to claim 1,
wherein the water-soluble component contains one or more components selected from a group consisting of a polyhydric alcohol, a high hydrophilic-lipophilic balance (HLB) surfactant, and a water-soluble oil agent.

3. The cosmetic product according to claim 1,
wherein a viscosity of the water-soluble component at 25° C. is approximately 40 mPa·s to 3,000 mPa·s.

4. The cosmetic product according to claim 1,
wherein an amount of the water-soluble component is approximately 1 to 30 mass % based on a total amount of the powder cosmetic layer.

5. The cosmetic product according to claim 1,
wherein the powder component contains spherical powder, and
wherein an amount of the spherical powder is approximately 1 to 30 mass % based on a total amount of the powder cosmetic layer.

6. The cosmetic product according to claim 1,
wherein an amount of the powder component is approximately 55 to 98.9 mass % based on a total amount of the powder cosmetic layer.

7. The cosmetic product according to claim 1,
wherein the water-soluble thickener contains one or more components selected from a group consisting of a clay mineral, a polysaccharide, and an acrylic thickener.

8. The cosmetic product according to claim 1,
wherein an amount of the water-soluble thickener is approximately 0.1 to 10 mass % based on a total amount of the powder cosmetic layer.

9. The cosmetic product according to claim 1,
wherein the support comprises a film, a woven fabric, or a foamed body.

10. A method for producing a cosmetic product, the method comprising:
attaching a slurry to a support, wherein the slurry contains a water-soluble component, a powder component, a water-soluble thickener, and water, and wherein the water-soluble component has a consistency of a liquid or a paste at 25° C.; and
forming a powder cosmetic layer containing the water-soluble component, the water-soluble thickener, and the powder component by removing at least part of the water from the slurry attached to the support.

11. The method for producing a cosmetic product according to claim 10,
wherein the water-soluble component contains one or more components selected from a group consisting of a polyhydric alcohol, a high BIB surfactant, and a water-soluble oil agent.

12. The method for producing a cosmetic product according to claim 10,
wherein a viscosity of the water-soluble component at 25° C. is approximately 40 mPa·s to 3,000 mPa·s.

13. The method for producing a cosmetic product according to claim 10,
wherein an amount of the water-soluble component is approximately 1 to 30 mass % based on a total amount of the powder cosmetic layer.

14. The method for producing a cosmetic product according to claim 10,
wherein the powder component contains spherical powder, and wherein a content of the spherical powder is approximately 1 to 30 mass % based on a total amount of the powder cosmetic layer.

15. The method for producing a cosmetic product according to claim 10,
wherein an amount of the powder component is approximately 55 to 98.9 mass % based on a total amount of the powder cosmetic layer.

16. The method for producing a cosmetic product according to claim 10,
wherein the water-soluble thickener contains one or more components selected from a group consisting of a clay mineral, a polysaccharide, and an acrylic thickener.

17. The method for producing a cosmetic product according to claim 10,
wherein a content of the water-soluble thickener is approximately 0.1 to 10 mass % based on a total amount of the powder cosmetic layer.

18. The method for producing a cosmetic product according to claim 10,
wherein the support comprises a film, a woven fabric, or a foamed body.
